(19) United States
(12) Patent Application Publication
(10) Pub. No.: US 2005/0119300 A1
(43) Pub. Date: Jun. 2, 2005

(54) Development of novel regulators of angiogenesis

(76) Inventor: Milton L Brown, Charlottesville, VA (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/501,021

(22) PCT Filed: Jan. 9, 2003
(86) PCT No.: PCT/US03/00593

Related U.S. Application Data
(60) Provisional application No. 60/347,372, filed on Jan. 11, 2002.

(51) Int. Cl. A61K 31/47; A61K 31/407; A61K 31/40; A61K 31/4035

(52) U.S. Cl. 514/309; 514/417; 514/411

(57) ABSTRACT

The present invention is directed to novel thalidomide derivative compounds that have activity as angiogenic and arteriogenic compounds. More particularly the compounds have the general structure: (I) wherein R [symbol] 3? is selected from the group consisting oH, C [symbol] 1?-C [symbol] 6? all, C [symbol] 1?-C [symbol] 6? alkenyl, C [symbol] 1?-C [symbol] 6? alkynyl and optionally substituted 5- or 6-membered rings. These compounds can be used to induce angiogenesis or arteriogenesis in a patient in need thereof.

### RELATED APPLICATIONS

This application claims priority under 35 U.S.C. § 119(e) to provisional patent application no. 60/347,372 filed Jan. 11, 2002 the disclosure of which is incorporated herein.

### FIELD OF THE INVENTION

The present invention is directed to novel phthalimides, and the use of such derivatives as therapeutic agents. More particularly, compositions comprising thalidomide and structurally related compounds are used as angiogenic agents.

### BACKGROUND OF THE INVENTION

Coronary heart disease (CHD) or narrowing of the coronary arteries caused 459,841 deaths in the United States in 1998, representing 20% of all mortality. Approximately every 29 seconds an American will suffer a coronary event, and about every minute someone will die from a CHD related disease, thus making CHD the single largest killer of American males and females. Because of the tremendous impact on human life and our health system, new treatment strategies are needed.

One promising area is angiogenesis research. The rapid development of angiogenic growth factor therapy for patients with advanced ischemic heart disease over the last 5 years offers hope of a new treatment strategy based on generation of new blood supply in the diseased heart. However, as the field of therapeutic coronary angiogenesis is maturing from basic and preclinical investigations to clinical trials, many new and presently unresolved issues are coming into focus. These include in-depth understanding of the biology of angiogenesis. Although scientists are far from realizing controlled growth of new blood vessels in humans, the American Heart Association recently defined challenges facing the development of therapeutic angiogenic agents for coronary disease. Even though much must be learned about potential adverse effects of angiogenic therapies, like the potential for stimulating retinopathy, leaky blood vessels/bleeding and carcinogenicity, clinical trials of angiogenic agents hold tremendous promise and commercial value.

Three different processes may contribute to the growth of new blood vessels: vasculogenesis, arteriogenesis, and angiogenesis. Vasculogenesis is the primary process responsible for growth of new vasculature during embryonic development and may play a yet-undefined role in mature adult tissues. It is characterized by differentiation of pluripotent endothelial cell precursors (hemangioblasts or similar cells) into endothelial cells that go on to form primitive blood vessels. Subsequent recruitment of other vascular cell types completes the process of vessel formation. The occurrence of vasculogenesis in mature organisms remains an unsettled issue. It is thought to be unlikely that this process contributes substantially to the new vessel development that occurs spontaneously in response to ischemia or inflammation as a response to growth factor stimulation.

Arteriogenesis refers to the appearance of new arteries possessing a fully developed tunica media. The process may involve maturation of pre-existing collaterals or may reflect de novo formation of mature vessels. Examples of arteriogenesis include formation of angiographically visible collaterals in patients with advanced obstructive coronary or peripheral vascular disease. All vascular cell types, including smooth muscle cells and pericytes, are involved. Arteriogenesis is the preferred type of neovascularization for purposes of restoring myocardial perfusion. Native arterial collateralization is a complex process that involves multiple levels of stimulators, inhibitors, and modulators. Therefore, the discovery of a drug molecule that induces therapeutic arteriogenesis, including the self-propagating cascade of proliferation, migration, chemotaxis would be useful.

Angiogenesis is the process responsible for formation of new vessels lacking developed media. Examples of angiogenesis include capillary proliferation in wound healing or along the border of myocardial infarction. Angiogenesis can be stimulated by a number of growth factors including fibroblast growth factor-2 (FGF-2) and vascular endothelial growth factor (VEGF). Further, insulin-like growth factor-I (IGF-I) can stimulate proliferation of these cells and can induce VEGF secretion. These growth factors appear to exert their effort directly on endothelial cells and reports indicate that these effects may be mediated through specific integrin molecules (αvβ3 or αvβ5).

The occurrence of both angiogenesis and arteriogenesis has been demonstrated conclusively in a variety of animal models, as well as in patients with coronary disease. Thus, insufficient angiogenesis may lead to tissue ischemia and failure. The recent discovery of novel angiogenic molecules has initiated efforts to improve tissue perfusion via therapeutic angiogenesis. However, rational design of novel treatment strategies and potential drugs mandates a better understanding of the molecular mechanisms of angiogenesis.

The role of a prime angiogenic candidate VEGF and its homologues, in physiological and pathological angiogenesis and its role in myocardial ischemia and heart failure has been the focus of recent interest. In addition, novel interactions between the junctional protein vascular endothelial associated proteins and VEGF is also in need of further investigation. Therefore the use of a small molecule agonist of VEGF could provide a potential novel therapeutic strategy.

Tissue ischemia is most likely not the only key stimulus in the initiation of an angiogenic response. Few patients demonstrate ongoing chronic myocardial ischemia, and most likely the majority of patients with diffuse multi-vessel disease do not develop tissue-level ischemia in the absence of provocation. Inflammation and shear stress may be much more important stimuli, and little angiogenesis takes place in the absence of inflammation. Suppression of inflammatory responses, due to pathophysiological processes or drug therapy, may adversely affect the ability to induce new vessel growth.

Myocyte necrosis stimulates inflammatory angiogenesis. Ischemic myocyte injury becomes irreversible, when there is a concomitant loss of capacity for reperfusion, termed the no-reflow phenomenon. Less severe temporary ischemia reduces the proportion of functional capillaries. Multiple mechanisms are involved in this microvascular stunning, including: reperfusion injury; leukocyte activation, adhesion and accumulation; and impaired endothelium-dependent vasodilation. Many of the microvascular changes are triggered by the inflammatory response to cell death and form part of a final common pathway in cardiac disease including ischemic heart disease. It is anticipated that stimulation of angiogenesis prior to myocyte necrosis in hypertrophy and control of leukocyte activity in ischemic heart disease could minimize myocyte loss or damage. The present invention is directed to the design, synthesis and evaluation of novel selective angiogenic compounds that have use in novel treatment strategies.

### SUMMARY OF THE INVENTION

The present invention is directed to compounds of the general formula: 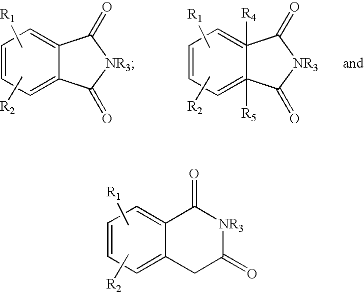
wherein R₁ and R₂ are independently selected from the group consisting of H, halo, alkyl, haloaIyl, —NR₆R₇, hydroxy and alkoxy, or R₁ and R₂ taken together; can form, with the adjacent ring, an optionally substituted 5- or 6-membered ring;

R₃ is selected from the group consisting of C₁-C₆ alkyl, C₁-C₆ alkenyl, C₁-C₆ alkynyl and optionally substituted 5- or 6-membered rings; and

R₄, R₅, R₆ and R₇ are independently H, or C₁-C₆ alkyl. The present invention also encompasses compositions comprising those compounds and the use of such compositions for stimulating angiogenesis and/or arteriogenesis.

### BRIEF DESCRIPTION OF THE DRAWINGS

FIGS. 1A-1D represents four graphs plotting the concentration of four respective compounds vs. inhibition of endothelial cell proliferation as measured by the ³H-thymidine uptake assay. FIG. 1A represents compound 1, FIG. 1A represents compound 2, FIG. 1A represents compound 3 and FIG. 1A represents compound 59.

FIG. 2 is a diagram of a series of compound 1 derivatives that are anticipated to exhibit endothelial cell proliferation activity.

FIG. 3 is a diagram of a series of compound 3 derivatives that are anticipated to exhibit endothelial cell proliferation activity.

FIG. 4 is a diagram of a series of compound 4 derivatives that are anticipated to exhibit endothelial cell proliferation activity.

### DETAILED DESCRIPTION OF THE INVENTION

In describing and claiming the invention, the following terminology will be used in accordance with the definitions set forth below.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” includes any of the standard pharmaceutical carriers, such as a phosphate buffered saline solution, water and emulsions such as an oil/water or water/oil emulsion, and various types of wetting agents.

As used herein, an “effective amount” means an amount sufficient to produce a selected effect. For example, an effective amount of the phthalimide derivative is an amount of the compound sufficient to increase growth rate of blood vessels in vivo or in vitro.

The general chemical terms used in the description of the compounds of the present invention have their usual meanings. For example, the term “alkyl” by itself or as part of another substituent means a straight or branched aliphatic chain having the stated number of carbon atoms.

The term “halo” includes bromo, chloro, fluoro, and iodo.

The term “haloalkyl” as used herein refers to a alkyl radical bearing at least one halogen substituent, for example, chloromethyl, fluoroethyl or trifluoromethyl and the like.

The term “C₁-Cₙ alkyl” wherein n is an integer, as used herein, refers to a branched or linear alkyl group having from one to the specified number of carbon atoms. Typically C₁-C₆ alkyl groups include, but are not limited to, methyl, ethyl, n-propyl, iso-propyl, butyl, iso-butyl, sec-butyl, tert-butyl, pentyl, hexyl and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

As used herein, the term “optionally substituted” refers to zero to four substituents, wherein the substituents are each independently selected. More preferredly, the term refers to zero to three independently selected substituents.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. Aryl groups (including bicyclic aryl groups) can be unsubstituted or substituted with one, two or three substituents independently selected from loweralkyl, haloalkyl, alkoxy, amino, alkylamino, dialkylamino, hydroxy, halo, and nitro. Substituted aryl includes aryl compounds having one or two C₁-C₆ alkyl, halo or amino substituents. The term (C₅-C₈ alkyl)aryl refers to any aryl group which is attached to the parent moiety via the alkyl group.

The term “C₃-Cₙ cycloalkyl” wherein n=4-8, represents cyclopropyl, cyclobutyl, cyclopentyl, cyclohexyl, cycloheptyl, and cyclooctyl.

The term “heterocyclic group” refers to a C₃-C₈ cycloalkyl group containing from one to three heteroatoms wherein the heteroatoms are selected from the group consisting of oxygen, sulfur, and nitrogen.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The term “fused aromatic” refers to a C₃-C₈ cycloalkyl group or C₃-C₈ heterocyclic group fused to one or more aryl groups. An especially preferred fused aromatic is a phenyl group fused to a heterocyclic group. The fused aromatic may be substituted at any position on the substituent including on the heterocyclic group and on the aromatic group.

The term “lower alkyl” as used herein refers to branched or straight chain alkyl groups comprising one to eight carbon atoms, including methyl, ethyl, propyl, isopropyl, n-butyl, t-butyl, neopentyl and the like.

The term, “parenteral” means not through the alimentary canal but by some other route such as subcutaneous, intramuscular, intraspinal, or intravenous.

As used herein, the term “treating” includes alleviating the symptoms associated with a specific disorder or condition and/or preventing or eliminating said symptoms.

The Invention

The present invention relates a novel series of phthalimide derivatives that are anticipated as having angiogenic activity. More particularly the present invention is directed to compounds having the general structure: 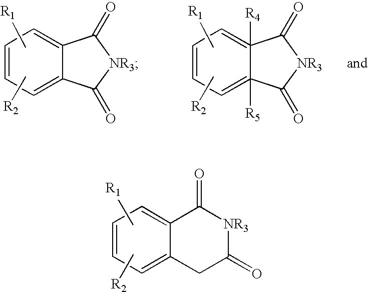
wherein R₁ and R₂ are independently selected from the group consisting of H, halo, alkyl, haloalkyl, —NR₆R₇, hydroxy and alkoxy, or R₁ and R₂ taken together can form, with the adjacent ring, an optionally substituted 5- or 6-membered ring, and more preferably form an optionally substituted C₅-C₆ cycloalkyl ring;

R₃ is selected from the group consisting of H, C₁-C₆ alkyl, C₁-C₆ alkenyl, C₁-C₆ alkynyl and optionally substituted 5- or 6-membered rings; and

R₄, R₅, R₆ and R₇ are independently H, or C₁-C₆ alkyl. When R₃ is a substituted 5- or 6-membered ring, the ring may be a cycloalkyl ring substituted with 1, 2 or 3 substituents selected from the group consisting of H, halo, alkyl, haloalkyl, —NR₆R₇, hydroxy, phthalimidyl and alkoxy. In accordance with one embodiment R₁ and R₂ are both H. In one embodiment R₁ and R₂ are both H, and R₃ is selected from the group consisting of C₁-C₆ alkyl and optionally substituted 5- or 6-membered rings.

In accordance with one embodiment an angiogenic and/or arteriogenic compound of the present invention has the general structure: 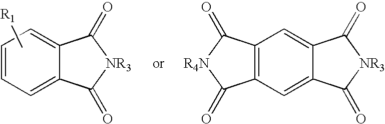
wherein R₁ is selected from the group consisting of H, halo, C₁-C₆ alkyl and C₁-C₆ alkoxy and R₃ and R₄ are independently selected from the group consisting of H, C₁-C₆ alkyl C₁-C₆ alkenyl, C₁-C₆ alkynyl and optionally substituted C₅-C₈ cycloalkyl. In one embodiment, R₁ is H, Cl, hydroxy, methoxy or methyl and R₃ and R₄ are independently H or C₁-C₃ alkyl. In one embodiment, R₁ is H or F and R₃ and R₄ are independently H or C₁-C₃ alkyl, and in one preferred embodiment R₁ is H and R₃ and R₄ are independently H or CH₃.

In one embodiment the angiogenic and/or arteriogenic compound has the general structure:

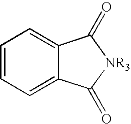

wherein R₃ is selected from the group consisting of H, C₁-C₆ alkyl, C₁-C₆ alkenyl, C₁-C₆ alkynyl, and in one embodiment R₃ is H or C₁-C₃ alkyl.

In one embodiment the antigenic compound has the structure

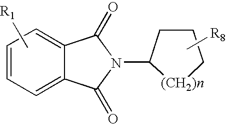

wherein R₁ is selected from the group consisting of H, halo and C₁-C₃ alkyl and R₈ is selected from the group consisting of H, halo, C₁-C₈ alkyl, —NH₂, hydroxy, C₁-C₈ alkoxy, aldehyde, COOH and COOCH₃, and n is an integer ranging from 1 to 4. In one embodiment R₁ is H, R₈ is H, halo, C₁-C₈ alkyl, —NH2, hydroxy, C₁-C₈ alkoxy, aldehyde, COOH or COOCH₃, and n is 1 or 2. In one embodiment R₁ is H, R₈ is H, Cl, F, hydroxy or methoxy, and n is 2. In another embodiment R₁ is H, R₈ is selected from the group consisting of H, Cl, F and hydroxy, and n is 2.

In accordance with one embodiment an angiogenic and/or arteriogenic compound is provided wherein the compounds has the general structure

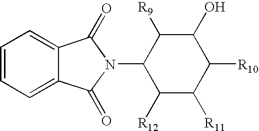

wherein R₉, R₁₀, R₁₁, and R₁₂ are independently selected from the group consisting of H, halo, C₁-C₆ alkyl, C₁-C₆ alkenyl, C₁-C₆ alkynyl, —NH₂, hydroxy, C₁-C₈ alkoxy, aldehyde, COOH and COOCH₃. In one embodiment R₉, R₁₀, R₁₁, and R₁₂ are independently selected from the group consisting of H, Cl, —NH₂, hydroxy, methoxy, aldehyde, COOH and COOCH₃. In one embodiment the compound has the structure 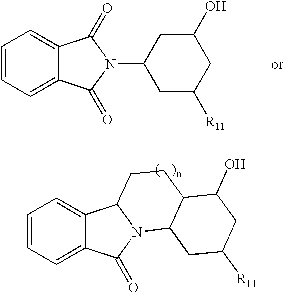
wherein n is an integer ranging from 0-3, and R₁₁ is selected from the group consisting of H, hydroxy and

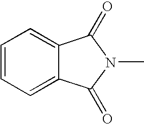

In accordance with another embodiment the angiogenic compound has the general structure: 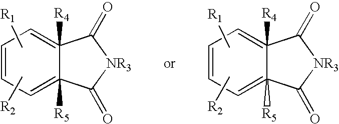
wherein R₁ and R₂ are independently selected from the group consisting of H, halo, alkyl, haloalkyl, —NR₆R₇, hydroxy and alkoxy, or R₁ and R₂ taken together, can form, with the adjacent ring, an optionally substituted 5- or 6-membered ring;

R₃ is selected from the group consisting of H, C₁-C₆ allyl, C₁-C₆ alkenyl, C₁-C₆ alkynyl and optionally substituted 5- or 6-membered rings; and

R₄ and R₅ are independently H, or C₁-C₆ alkyl. In accordance with one embodiment R₁ and R₂ are both H, R₃is H, C₁-C₆ alkyl, C₁-C₆ alkenyl or C₁-C₆ alkynyl and R₄ and R₅ are independently H, or C₁-C₃ alkyl. In one embodiment, R₁ and R₂ are both H, R₃ is H or C₁-C₄ alkyl and R₄ and R₅ are independently H or CH₃.

One set of compounds suitable for use in accordance with the present invention includes compounds have the following structures:

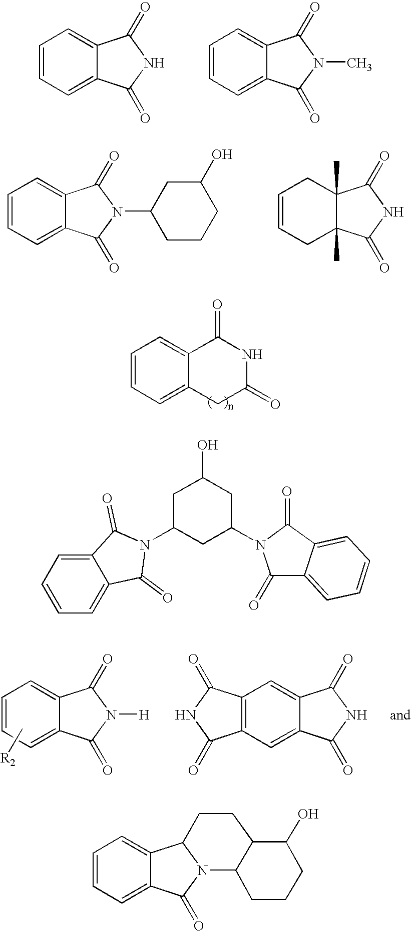

wherein R₂ is selected from the group consisting of H, halo, C₁-C₄ alkyl, halo(C₁-C₄)alkyl and C₁-C₄ alkoxy and n is an integer ranging from 1 to 4, and more preferably n is 1 or 2.

In accordance with one embodiment the angiogenic/arteriogenic compounds of the present invention can be formulated as pharmaceutical compositions by combining the compounds with one or more pharmaceutically acceptable carriers. Such pharmaceutically compositions can be utilized as angiogenic/arteriogenic compositions. The compounds can be administered either orally or parenterally. In one embodiment a composition comprising the thalidomide derivative of the present invention are administered locally by injection or implantable time release device. When administered orally, the compounds can be administered as a liquid solution, powder, tablet, capsule or lozenge. The compounds can be used in combination with one or more conventional pharmaceutical additive or excipients used in the preparation of tablets, capsules, lozenges and other orally administrable forms. When administered parenterally, and more preferably by intravenous injection, the derivatives of the present invention can be admixed with saline solutions and/or conventional IV solutions.

In accordance with one embodiment a method for inducing angiogenesis or arteriogenesis is provided. The method comprises the steps of contacting endothelial cells with a compound selected from the group consisting of

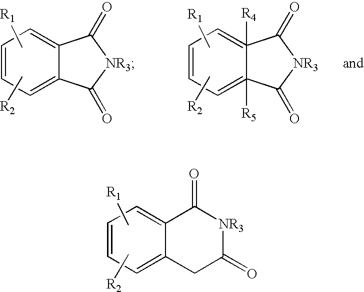

wherein R₁ and R₂ are independently selected from the group consisting of H, halo, alkyl, haloalkyl, —NR₆R₇, hydroxy and alkoxy, or R₁ and R₂ taken together; can form, with the adjacent ring, an optionally substituted 5- or 6-membered ring;

R₃ is selected from the group consisting of H, C₁-C₆ alkyl, C₁-C₆ alkenyl, C₁-C₆ alkynyl and optionally substituted 5- or 6-membered rings; and

R₄, R₅, R₆ and R₇ are independently H, or C₁-C₆ alkyl. In accordance with one embodiment R₁ and R₂ are both H.

In accordance with another embodiment the thalidomide derivatives of the present invention are used to induce arteriogenesis and/or angiogenesis in a warm blooded vertebrate, and more preferably in a mammalian species such as humans. In accordance with this embodiment, the present compounds can be used to treat a variety of conditions or disease states that are characterized by an insufficient vascularization. The method comprises administering a pharmaceutical composition comprising compound selected from the group consisting of

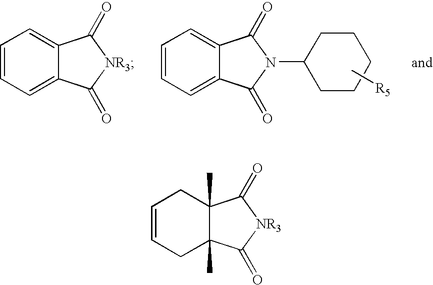

wherein R₃ is H or C₁-C₃ alkyl; and

R₅ is selected from the group consisting of H, hydroxy and C₁-C₃ alkyl, and ore preferably R₃ is H or CH₃ and R is —OH. In one preferred embodiment the compound is selected from the group consisting of

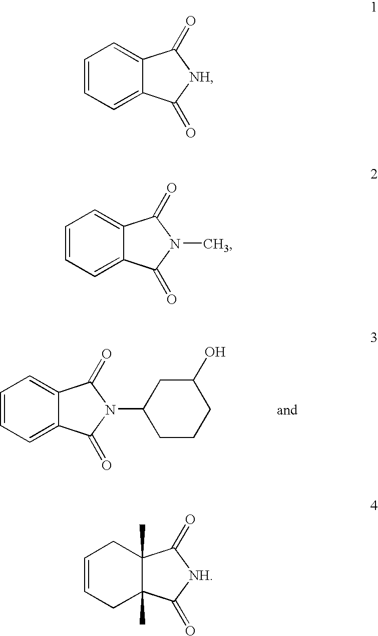

As described in Example 2, compounds 14 have been demonstrated to have angiogenic activity. Compounds 1-4 are structurally related to the anti-angiogenic compound thalidomide: 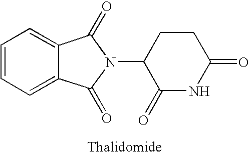
Thus it is quite possible that the compounds of the present invention could be interacting as an activator to the same site of thalidomide inhibitory actions. Thalidomide has been reported as having significant anti-angiogenic efficacy against vascular endothelial growth factor (VEGF) and basic fibroblast growth factor (bFGF) induced angiogenesis. Thalidomide and a thalidomide analog (cc-1069) also demonstrated ability to inhibit the in vitro proliferation of endothelial cells (cells which make up the vascular system). Although a limited number of analogs have been reported, there appears to exist developing SAR for anti-angiogenesis. Further studies reveal that the S(−)-enantiomer of thalidomide has potent anti-angiogenic activity in both VEGF-induced and bFGF-induced corneal neovascularization. Taken together, the SAR and enantioselective inhibition strongly supports a receptor mechanism of action.

Because of the structural similarity of the present analogs (proliferators) to thalidomide (anti-proliferative), it is anticipated that the present compounds may have proliferative effects through growth factor VEGF and bFGF receptors and thus these receptors make putative targets for mechanistic evaluation. Accordingly, the compounds of the present invention can be combined and used in conjunction with such growth factors. Other equally important targets to investigate include growth factors (IGF-I, FGF-2) and/or targets from the growth factor stimulation pathway including the integrin genes themselves.

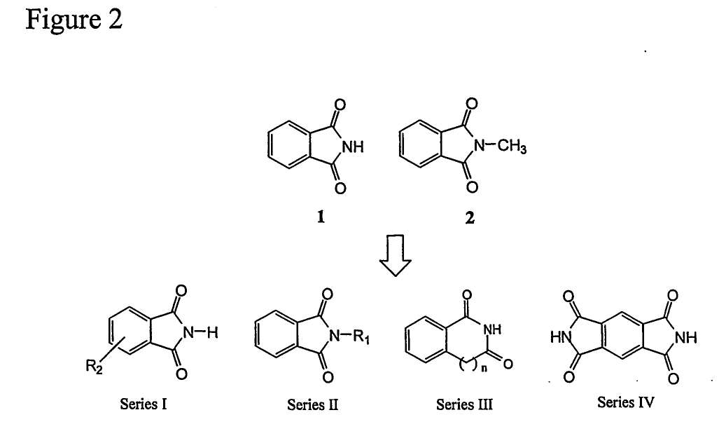

Based on the activity demonstrated for compounds 1-4, a series of structurally similar compounds can be prepared using traditional medicinal chemistry transformations. Series I-IV (See FIG. 2) will explore the pharmacophore of compound 1. Series I will investigate the effects of phenyl ring substitution on proliferation by ortho, meta and para substitutions of Cl, OCH₃, OH and CH₃. Series II represents evaluation of effects of imide substitutions on proliferation. Preliminary evidence suggest that alkyl groups are tolerated at this position and thus analogs will be prepare wherein R₃=ethyl, propyl, allyl and alkynyl. Series III involves ring expansion of the phthalimide ring to isoquinoline (n=1) and larger ring sizes (n=2 and 3) to optimize the appropriate ring system. Series IV represents a novel diphthalimide (duplication).

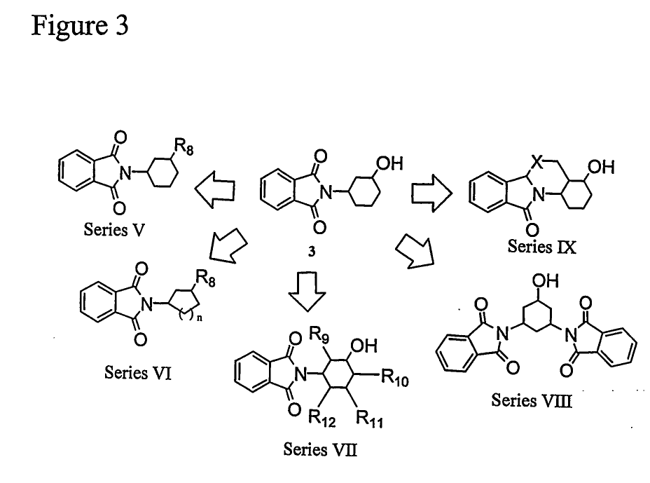

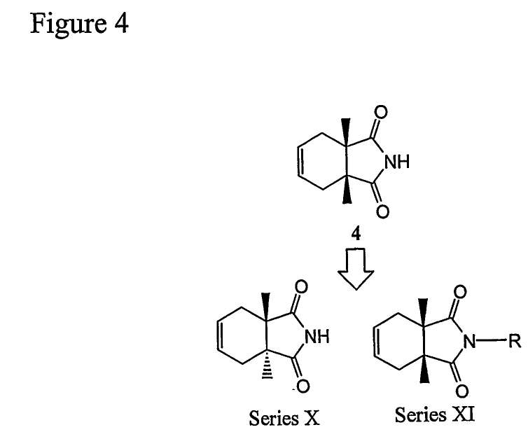

Series V-IX (see FIG. 3) were designed to optimize analog 3. Series V represent functional group replacements of 3 (substituents used include Cl, OCH₃, aldehyde, NH₂ and COOH and COOCH₃). Series VI will investigate effects of ring size on proliferation and series VII effects of hydroxyl substitutions on proliferation. Series VIII represents a dual inhibitor of compound 3. Overall total of 66 compounds will be synthesized that explore the important structural features of leads 1-4.

Finally, the activity of the present compounds for proliferational effects on aorta we will be investigated. Previously, rings of rat aorta embedded in gels of fibrin or collagen and cultured in MCDB 131( an optimized growth medium for microvascular endothelial cells) generate branching microvessels in the absence of serum or other soluble protein supplements. The angiogenic response is self-limited and can be quantitated by counting the newly formed microvessels daily in the living cultures. The microvascular growth curves are characteristic for each gel. Growth of microvessels in collagen gel peaks at the end of the 1st week and is followed by a rapid regression in the 2nd week. This method represents a facile way of measuring effects of our compounds on the angiogenic stimulation of the aortic microvasculature.

In accordance with one embodiment a method is provided for inducing arteriogenesis and/or angiogenesis in a warm blooded vertebrate, and more preferably in a mammalian species such as humans. The method comprises administering a pharmaceutical composition comprising a compound having the general structure: 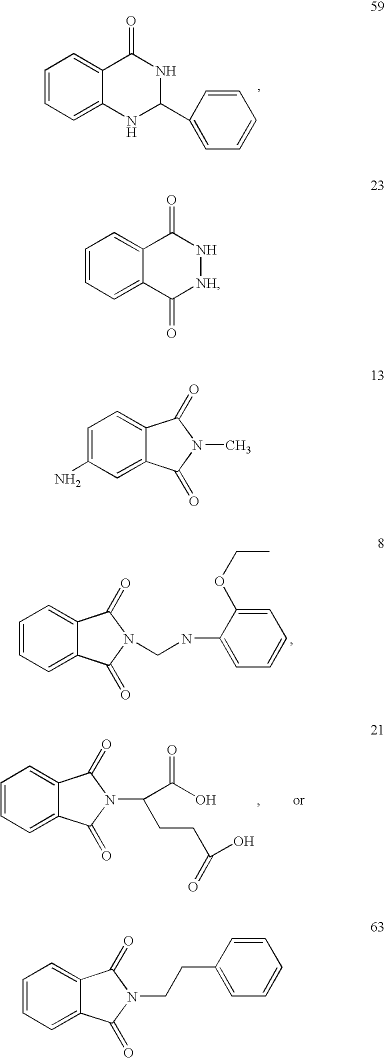
in an amount that produces an in vivo concentration of less than 100 uM of the active compound.

### EXAMPLE 1

### Synthetic Schemes for Preparing the Claimed Compounds

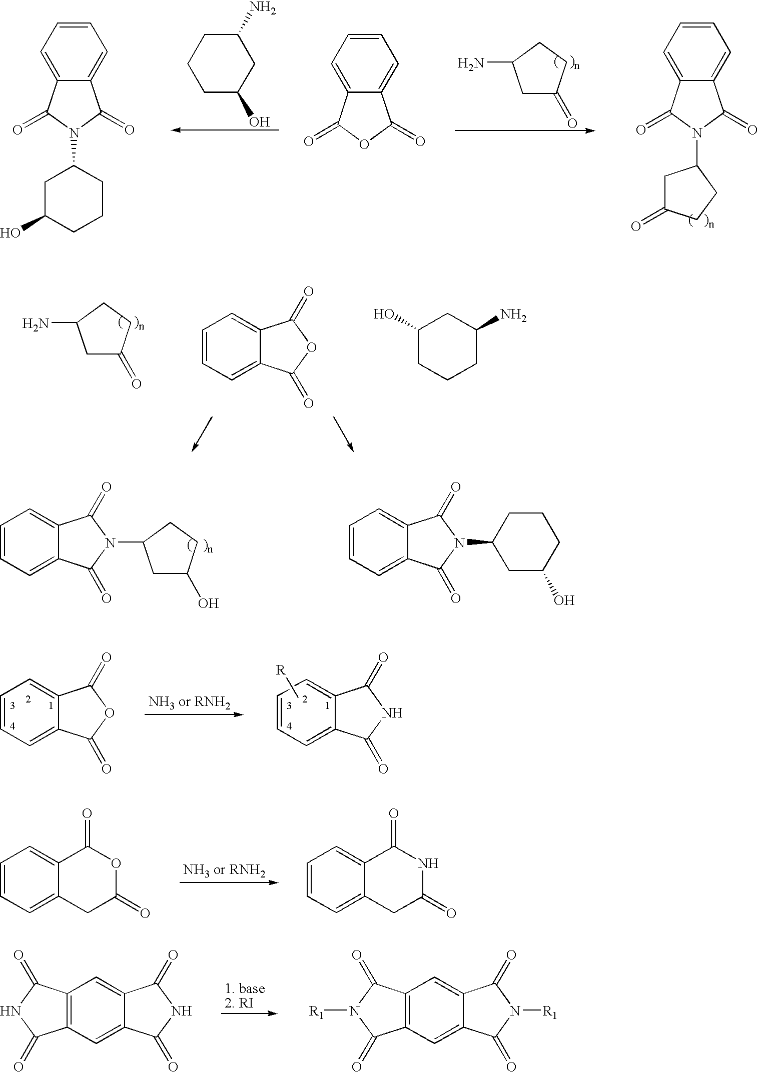

### EXAMPLE 2

### Isolation of Novel Angiogenic Compounds

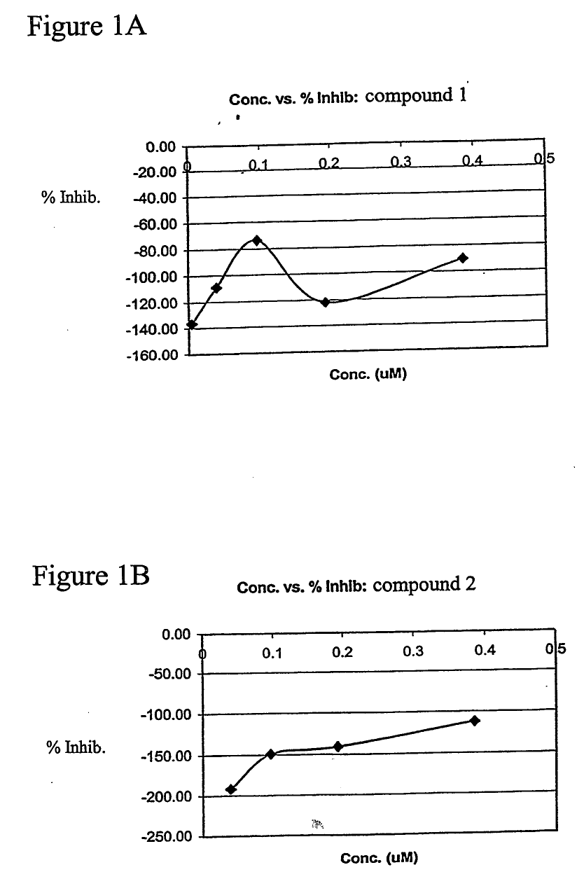

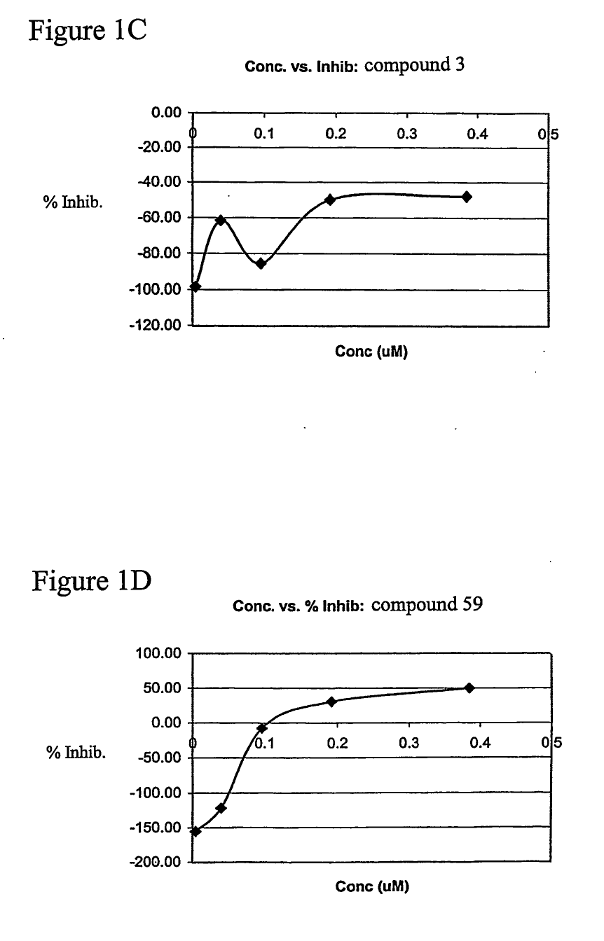

In pursuit of novel angiogenic compounds, structural types 1-4 were found to induce endothelial cell proliferation as a measure of ³H-thymidine uptake. Briefly, human vascular endothelial cells (HUVECS) were cultured to peri-confluence (80%) in 20% serum and treated with thalidomide (standard) or its analog (40-400 M). After 20 h, [³H]-thymidine (2 μCi/ml) was added to the culture medium for 2-4 h. The [³H]-thymidine incorporation was stopped with ice-cold PBS (3 washes) and the cells were incubated with cold 10% trichloroacetic acid (TCA) for 10 min at 4C. The cells were further incubated with TCA at room temperature for 10 min and washed three times with PBS. The cells were solubilized overnight with 1N NaOH and neutralized with an equivalent amount of 1N HCl before radioactivity was determined. The anti-proliferative activity of thalidomide or the proliferative activities of analogs 1-4 were computed as a percent inhibition of HUVECS mitogenic response to 20% serum (fetal calf serum). The results of the screen of about 40 analogs revealed compounds 1-4 to promote increased proliferation of endothelial cells (see FIG. 1 and Table 1A & 1B) as compared to thalidomide which demonstrated significant inhibition of endothelial cell proliferation (IC₅₀=185 25 M).

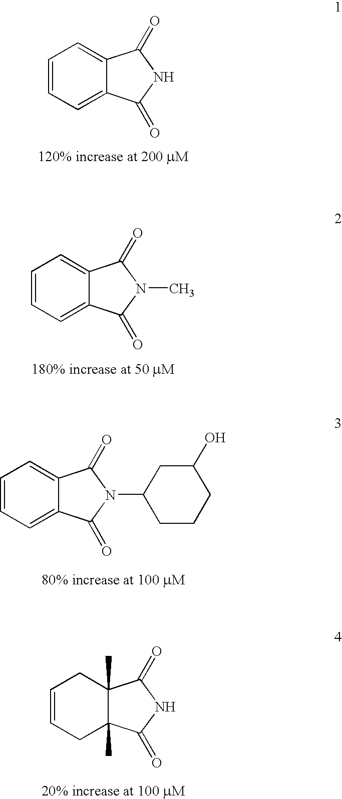

TABLE 1A

| | | | | | | % Inhibition vs |
|---|---|---|---|---|---|---|
| | Well 1 | Well 2 | Well 3 | Well 4 | Average | 10% Serum |
| 10% | 1283.697 | 1919.027 | 1727.56 | 1757.906 | 1672.048 | 0 |
| Serum, | | | | | | |
| DMSO | | | | | | |
| STD | 141.5772 | 255.4687 | 210.2551 | 271.6354 | 219.7341 | 86.85838246 |
| (385 uM) | | | | | | |
| STD | 198.4536 | 273.8429 | 272.3789 | 235.6478 | 245.0808 | 85.34247382 |
| (289 uM) | | | | | | |
| STD | 255.2577 | 435.4396 | 336.7686 | 411.8425 | 359.8271 | 78.4798518 |
| (192.5 uM) | | | | | | |
| STD | 1736.688 | 850.094 | 1083.54 | 985.3011 | 1163.906 | 30.39038813 |
| (96 uM) | | | | | | |
| STD | 1835.938 | 2018.496 | 4751.43 | 2194.99 | 2700.214 | −61.4914349 |
| (38.5 uM) | | | | | | |
| Cmpd 13 | * | 1391.864 | 1332.559 | 1408.206 | 1377.543 | 17.61340512 |
| (385 uM) | | | | | | |
| Cmpd 13 | 1866.398 | 1504.124 | 1485.233 | 1538.841 | 1598.649 | 4.389737732 |
| (289 uM) | | | | | | |
| Cmpd 13 | 1408.926 | 1277.343 | 4210.35 | 1886.464 | 2195.771 | −31.32227105 |
| (192.5 uM) | | | | | | |
| Cmpd 13 | 1316.135 | 1678.165 | 1623.241 | 1913.034 | 1632.644 | 2.356616663 |
| (96 uM) | | | | | | |
| Cmpd 13 | 1754.12 | 1696.046 | 1625.193 | 1893.992 | 1742.338 | −6.718795818 |
| (38.5 uM) | | | | | | |
| Cmpd 21 | 1325.84 | 1890.539 | 2214.16 | 2603.942 | 2008.62 | −20.12937731 |
| (385 uM) | | | | | | |
| Cmpd 21 | 1725.392 | 1749.762 | 3035.108 | 2328.617 | 2209.72 | −32.15651768 |
| (289 uM) | | | | | | |
| Cmpd 21 | 2155.062 | 1562.824 | 2205.943 | 2241.275 | 2041.276 | −22.08241692 |
| (192.5 uM) | | | | | | |
| Cmpd 21 | 1155.747 | 1347.344 | 1111.246 | 1576.482 | 1297.705 | 22.38828442 |
| (96 uM) | | | | | | |
| Cmpd 21 | 1478.661 | 1627.736 | 1527.77 | 2195.756 | 1707.481 | −2.119153314 |
| (38.5 uM) | | | | | | |
| Cmpd 23 | 1330.199 | 2033.188 | 1584.499 | 2777.121 | 1931.252 | −15.50220613 |
| (385 uM) | | | | | | |
| Cmpd 23 | 2278.233 | 1530.61 | 2151.177 | 1180.738 | 1785.19 | −6.766673794 |
| (289 uM) | | | | | | |
| Cmpd 23 | 2361.087 | 4603.669 | 1888.869 | 1994.525 | 2712.038 | −62.19859185 |
| (192.5 uM) | | | | | | |
| Cmpd 23 | 2175.65 | 1895.757 | 1415.829 | 1483.496 | 1742.683 | −4.22449123 |
| (96 uM) | | | | | | |
| Cmpd 23 | 2041.647 | 1605.488 | 1852.148 | 1614.298 | 1778.395 | −6.360330672 |
| (38.5 uM) | | | | | | |

TABLE 1B

| | | | | | | % Inhibition |
|---|---|---|---|---|---|---|
| | | | | | | vs |
| | Well 1 | Well 2 | Well 3 | Well 4 | Average | 10% Serum |
| 10% | 14383 | 23393 | 19729 | 30981 | 22122 | 0.0 |
| Serum, | | | | | | |
| DMSO | | | | | | |
| Cmpd 8 | 725 | 362 | 382 | 351 | 455 | 97.9 |
| (192 uM) | | | | | | |
| Cmpd 8 | 3115 | 2106 | 540 | 556 | 1579 | 92.9 |
| (146 uM) | | | | | | |
| Cmpd 8 | 1390 | 2204 | 1485 | 755 | 1459 | 93.4 |
| (96 uM) | | | | | | |
| Cmpd 8 | 21752 | 14878 | 15721 | 17726 | 17519 | 20.8 |
| (77 uM) | | | | | | |
| Cmpd 8 | 31737 | 18013 | 39063 | 34191 | 30751 | −39.0 |
| (38.5 uM) | | | | | | |
| Cmpd 63 | 18553 | 11513 | 11633 | 18783 | 15121 | 31.6 |
| (192 uM) | | | | | | |
| Cmpd 63 | 25377 | 10563 | 13008 | 14764 | 15928 | 28.0 |
| (146 uM) | | | | | | |
| Cmpd 63 | 30451 | 25638 | 22013 | 17833 | 23984 | −8.4 |
| (96 uM) | | | | | | |
| Cmpd 63 | 22055 | 19183 | 25296 | 21076 | 21903 | 1.0 |
| (77 uM) | | | | | | |
| Cmpd 63 | 23933 | 27216 | 27730 | 27219 | 26525 | −19.9 |
| (38.5 uM) | | | | | | |

1. A pharmaceutical composition comprising a compound selected from the group consisting of 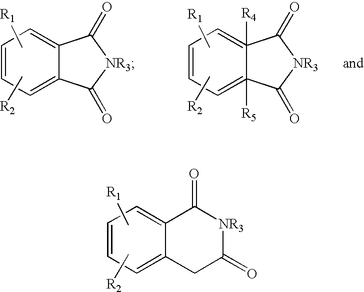
wherein R₁ and R₂ are independently selected from the group consisting of H, halo, alkyl, haloalkyl, —NR₆R₇, hydroxy and alkoxy, or R₁ and R₂ taken together; can form, with the adjacent ring, an optionally substituted 5- or 6-membered ring;
R₃ is selected from the group consisting of H, C₁-C₆ alkyl, C₁-C₆ alkenyl, C₁-C₆ alkynyl and optionally substituted 5- or 6-membered rings; and
R₄ and R₅ are independently H, or C₁-C₆ alkyl; and
a pharmaceutically acceptable carrier.

2. The composition of claim 1 wherein R₁ is selected from the group consisting of H, halo, alkyl, haloalkyl, —NH₂, hydroxy and alkoxy;
R₂ is H;
R₃ is selected from the group consisting of H, C₁-C₆ alkyl, C₁-C₆ alkenyl, C₁-C₆ alkynyl and optionally substituted C₅-C₆ cycloalkyl; and
R₄, R₅, R₆ and R₇ are independently H, or C₁-C₆ alkyl.

3. The composition of claim 2 wherein the compound is selected from the group consisting of 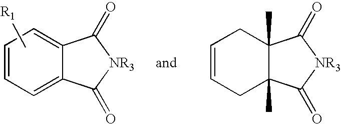
wherein R₁ is H or halo; and
R₃ is selected from the group consisting of H, C₁-C₃ allyl, C₁-C₃ alkenyl, C₁-C₃ alkynyl and optionally substituted C₅-C₆ cycloalkyl.

4. The composition of claims 2 or 3 wherein R₁ is H or F.

5. The composition of claim 1 wherein the compound is selected from the group consisting of 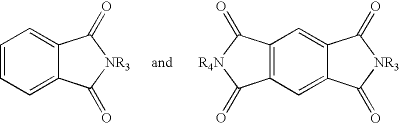
wherein R₃ and R₄ are independently selected from the group consisting of H, C₁-C₃ alkyl, C₁-C₃ alkenyl, C₁-C₃ alkynyl and optionally substituted C₅-C₆ cycloalkyl.

6. The composition of claim 1 wherein the compound is selected from the group consisting of 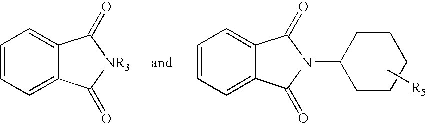
wherein R₃ is H or C₁-C₂ alkyl; and
R₅ is selected from the group consisting of H, —NH₂, hydroxy and C₁-C₃ alkoxy.

7. The composition of claim 6 wherein R₅ is H or hydroxy and R₃ is H or CH₃.

8. A method of inducing angiogenesis in a warm blooded vertebrate, said method comprising the steps of administering to said vertebrate a compound selected from the group consisting of 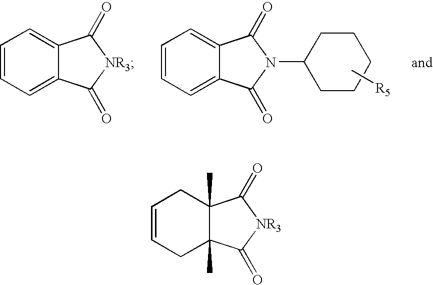
wherein R₃ is H or C₁-C₃ alkyl; and
R₅ is selected from the group consisting of H, hydroxy and C₁-C₃ alkyl.

9. The method of claim 8 wherein the compound is selected from the group consisting of

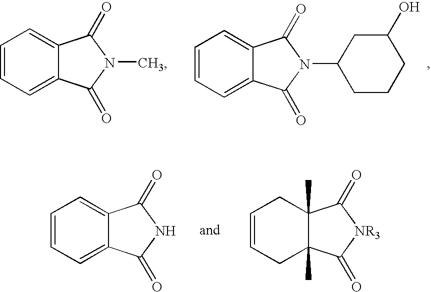

10. A method of promoting wound healing in a warm blooded vertebrate, said method comprising the steps of administering to said vertebrate a composition comprising a compound selected from the group consisting of 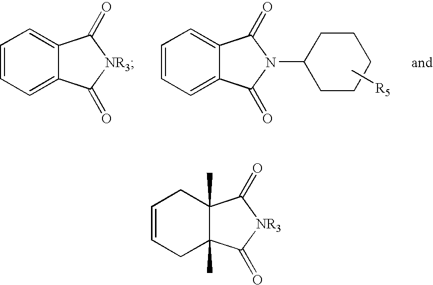
wherein R₃ is H or C₁-C₃ alkyl; and
R₅ is selected from the group consisting of H, —NH₂, hydroxy and C₁-C₃ alkyl.

11. A pharmaceutical composition comprising an angiogenic compound selected from the group consisting of 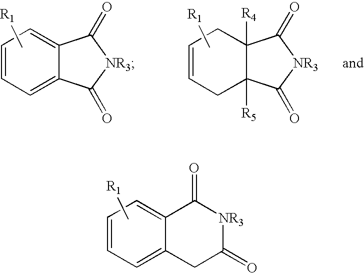
wherein R₁ is selected from the group consisting of H, halo, alkyl, haloalkyl, —NH₂, hydroxy and alkoxy;
R₃ is selected from the group consisting of H, C₁-C₆ alkyl, C₁-C₆ alkenyl, C₁-C₆ alkynyl and optionally substituted 5- or 6-membered rings; and
R₄ and R₅ are independently H, or C₁-C₆ alkyl; and
a pharmaceutically acceptable carrier.

12. The composition of claim 11 wherein the compound is selected from the group consisting of 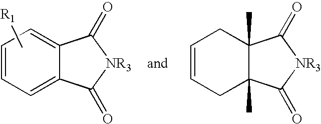
wherein R₁ is H or halo; and
R₃ is selected from the group consisting of H, C₁-C₃ alkyl, C₁-C₃ alkenyl, C₁-C₃ alkynyl and optionally substituted C₅-C₆ cycloalkyl.

13. The composition of claim 12 wherein R₁ is H and R₃ is selected from the group consisting of H, C₁-C₃ alkyl, C₁-C₃ alkenyl, C₁-C₃ alkynyl and 2-hydroxy cyclohexane.
